(19) United States
(12) Patent Application Publication
(10) Pub. No.: US 2023/0020166 A1
(43) Pub. Date: Jan. 19, 2023

(54) EFFICIENT QUANTUM CHEMISTRY SIMULATION USING GATE-BASED QUBIT QUANTUM DEVICES

(71) Applicant: QU & CO CHEMISTRY B.V., Amsterdam (NL)

(72) Inventors: Vincent Elfving, Amsterdam (NL); Jose Gamez, Leverkusen (DE); Christian Gogolin, Leverkusen (DE)

(21) Appl. No.: 17/777,730

(22) PCT Filed: Nov. 18, 2020
(86) PCT No.: PCT/EP2020/082603

§ 371 Date: May 18, 2022

(30) Foreign Application Priority Data
Nov. 18, 2019 (EP) 19209818.4

(51) Int. Cl. G16C 10/00; G16C 20/30

(52) U.S. Cl. CPC G16C 10/00; G16C 20/30

(57) ABSTRACT

A method for simulating a quantum chemistry system comprises determining a hard-core bosonic Hamiltonian describing the quantum chemistry system, the Hamiltonian model restricting the electronic states to electron singlet state configurations; determining a “paired-electron unitary coupled cluster with double excitations” (pUCCD) ansatz, the ansatz being restricted to paired-electron configurations; mapping the pUCCD ansatz to qubit operations of a quantum circuit that comprises a set of qubits and gates for enabling pairs of qubits to interact with each other: and, determining a trial state on the quantum circuit by applying the qubit operations defined by the mapped pUCCD ansatz to the qubits; and, determining an energy of the quantum chemistry system based on the trial state and the restricted Hamiltonian, grouping the Hamiltonian terms into three sets of operators which can be measured simultaneously; and, an error-mitigation technique, based on post-selection of the quantum measurements with the known particle number.

### CROSS-REFERENCE TO RELATED APPLICATIONS

The present application is a Section 371 National Stage Application of International Application No. PCT/EP2020/082603, filed Nov. 18, 2020 and published as WO 2021/099428 A1 on May 27, 2021, in English, and further claims priority to European patent application Ser. No. 19209818.4, filed Nov. 18, 2019.

### FIELD OF THE INVENTION

The disclosure relates to efficient simulation of quantum chemistry using gate-based qubit quantum devices and, in particular, though not exclusively, to methods and systems for simulating quantum chemistry using gate-based qubit quantum devices, and a computer program product enabling gate-based qubit quantum devices to perform such simulation methods.

### BACKGROUND OF THE INVENTION

Techniques for simulating quantum chemistry on classical computers can be categorized roughly into full configuration interaction (FCI), density functional theory (DFT) and coupled clusters (CC) techniques. Reference is made to Yudong Cao et al., Quantum Chemistry in the Age of Quantum Computing, Chemical Reviews Article ASAP, DOI: 10.1021/acs.chemrev.8b00803, arXiv 1812.09976, 2019 for an extensive review of the field of quantum chemistry simulation.

An FCI solution of an electronic structure Hamiltonian requires exponential time on a classical computer. DFT techniques make severe approximations in order to approximately solve the spectra, even for large systems, but so that a high level of accuracy cannot be attained. Coupled Cluster (CC) techniques achieve a relatively good accuracy at polynomial computation costs. Typical state-of-the-art Coupled Cluster with Single and Double excitations, CCSD(T) techniques, without applying severe approximations which reduce accuracy, scale as [symbol] (N⁶)- [symbol] (N⁸) (the order depending on the particular form) where N is the number of spin-orbitals in the problem.

State-of-the-art protocols for simulating quantum chemistry on a quantum computer can be roughly categorized into near-term NISQ (Noisy Intermediate Scale Quantum) algorithms and future FTQC (Fault Tolerant Quantum Computing) algorithms. Simulating ground state energies on a FTOC system can be done with a variety of algorithms, from which (various variants on) the so-called Quantum Phase Estimation (QPE) algorithm is a principal candidate. QPE may in principle simulate spectra and dynamics of chemistry Hamiltonians to arbitrary accuracy; however, the coherence requirements are much more stringent than present-day quantum devices allow for.

Conversely, in the NISQ era, the most widely used algorithm for ground-state estimation is the Unitary Coupled Cluster with Single and Double excitations—Variational Quantum Eigensolver (UCCSD-VQE) scheme. UCCSD-VQE is a variational technique in which first a quantum device is prepared in a state that approximates to the ground state, such as a Hartree-Fock state, after which a suitable ansatz is applied to it in a variational hybrid quantum-classical approach in order to converge to a good approximation of the ground state. The UCCSD is a unitary variant of the classical CCSD protocol which is expected to give improved accuracy.

Using a Gaussian orbital basis decomposition of the Hamiltonian in second-quantization, the computational quantum circuit depth scaling of the UCCSD protocol is [symbol] (N⁴) in state-of-the-art proposals, while the number of measurements has a pre-factor between [symbol] (N³)- [symbol] (N⁴). Babbush et al., describe in their article Low-Depth Quantum Simulation of Materials, Phys. Rev. X 8, 011044, 2018 the use of a particular dual to a plane-wave basis decomposition for the Hamiltonian, in combination with the use of a Fermionic swap-network as described by Kivlichan et al. in their article Quantum Simulation of Electronic Structure with Linear Depth and Connectivity, Phys. Rev. Lett. 120, 110501 2018. Such swap network allows for implementing Trotterized operator evolution on a linear array of qubits with depth scaling linear with the array length. In that case, the primitive circuit depth can be as low as [symbol] (N) with a number of measurements having a pre-factor of [symbol] (N²). However, this scaling requires a fermionic fast Fourier transform (FFFT) sub-routine as described in the above-referenced article by Babbush et al., which may have prohibitive coherence requirements for near-term devices. Also, a periodic basis set may be ill-suited for simulating molecular chemistry, requiring a large pre-factor scaling which prohibits near-term quantum device implementation, and a chemistry-inspired quantum Ansatz in a periodic basis set has not yet been identified.

Hence, from the above, it follows that there is therefore a need in the art for improved methods and systems for simulating quantum chemistry using gate-based qubit quantum devices. In particular, there is a need in the art to improve on the computational complexity scaling for simulating the Hamiltonian spectra of quantum chemistry problems using gate-based qubit quantum devices, as well as to make the quantum part of the algorithm more suitable for realistic quantum hardware which exhibits noise and has finite runtime restrictions.

### SUMMARY OF THE INVENTION

It is an objective of the embodiments in this disclosure to reduce or eliminate at least three of the drawbacks known in the prior art. In particular, it is an object of the embodiments in this application to improve on the computational complexity scaling (in terms of the total wall-clock time) for simulating the Hamiltonian spectra of a quantum chemistry problem. A further object of the embodiments is the simulation of much larger molecular systems than previously, now effectively simulable using current-day quantum hardware. It is a yet further object of the embodiments to reduce the primitive gate-depth requirement of the relevant quantum circuit, as compared to conventional approaches, in order to stay within the practical limits set by quantum decoherence of the quantum hardware operations. It is also an object of the embodiments to simplify and accelerate the measurement scheme used on the quantum device. It is also an object of the embodiments to apply a cost-effective error mitigation scheme to compensate for the shortcomings of quantum hardware devices. It is also an object of the embodiments to reduce the gate-depth requirement of the FTQC implementation of this scheme, in order to reduce the total runtime requirements and error correction resource demands.

The embodiments aim to simulate molecular chemistry in a restricted Hilbert-space with a novel mapping and simulation technique, which effectively reduces the computational complexity by a polynomial factor. Furthermore, the simulation can be executed with a two-fold increase in simulable system size or basis set, using the same quantum hardware dimensions as conventional. This allows for increased accuracy, given a fixed set of quantum resources.

In an aspect, the invention may relate to a method for simulating a quantum chemistry system comprising: determining a Hamiltonian describing the quantum chemistry system, the Hamiltonian being restricted to molecular orbitals that are occupied or not occupied by electron pairs; determining a paired-electron unitary coupled cluster with double excitations (pUCCD) ansatz, the ansatz being restricted to molecular orbitals that are occupied or not occupied by electron pairs; mapping the pUCCD ansatz to qubit operations of a quantum circuit, the quantum circuit comprising a set of qubits and gates for enabling pairs of qubits to interact with each other; and, determining a trial state on the quantum circuit by applying the qubit operations defined by the mapped pUCCD ansatz to the qubits; and, determining energy of the energy of the quantum chemistry system based on the trial state and the restricted Hamiltonian.

The invention may also relate to method for simulating a quantum chemistry system using a data processing system comprising a classical computer connected to a quantum computer. In an embodiment, the method may comprise: receiving or determining, by the classical computer, information on a Hamiltonian describing the quantum chemistry system, the Hamiltonian being a hard-core bosonic Hamiltonian, which restricted to electron singlet state configurations; receiving or determining, by the classical computer, information on a paired-electron unitary coupled cluster with double excitations (pUCCD) ansatz, the ansatz being restricted to electron singlet state configurations; transforming, by the classical computer, the pUCCD ansatz into a quantum circuit, the quantum circuit representing a sequence of qubit operations; executing, by the quantum computer, the quantum circuit, the execution including applying the sequence of gate operations to qubits of the quantum computer; receiving, by the classical computer, a trial state, the trial state including measured expectation values of the Hamiltonian; and, determining an energy of the quantum chemistry system based on the trial state.

The restricted Hamiltonian, a hard-core bosonic Hamiltonian, can be directly be mapped to qubits of quantum computer which follows. This follows directly from its matching commutation relations. This results in a very efficient read-out of the quantum circuit. The restriction thus provided a very hardware efficient quantum algorithm, including a reduction in scaling which is in stark contrast to the original fermionic Hamiltonian mapping to qubits, which even after grouping, results in N⁴ mutually-commuting terms. Fermionic Hamiltonians known from the prior art, cannot be mapped to qubits directly/naturally, whereas the hard-core bosonic Hamiltonian is directly supported on qubits due to the matching commutation relations of the shared SU(2) group. Fermionic Hamiltonians can be mapped to qubits using for example the Jordan-Wigner transformation. This transformation maps each fermionic operator to a string of Pauli operators (or Pauli ‘words’), with terms like X/Y/Z spread around, which support on several qubits in a chain potentially covering the whole quantum processor. This introduces huge complexity in the measurement scheme, as each qubit would need to be rotated to a particular basis and the terms cannot be grouped into a lower number of shared tensor-product bases than O(N⁴).

Thus, while conventional un-restricted second-quantization Fermionic Hamiltonians have [symbol] - [symbol] (depending on optimal sorting capabilities) non-commuting sets of Hamiltonian terms which need to be measured independently, the restricted Hamiltonian scheme described here has [symbol] terms which can be sorted into just three non-commuting sets of terms which may be measured simultaneously, which means a constant [symbol] (1) impact on the overall time scaling, instead of [symbol] (N³)- [symbol] (N⁴). This results in a drastic polynomial improvement in Hamiltonian averaging contribution to the overall quantum simulation time, the total runtime of the simulation. Beside the gain in runtime, a significant reduction in the complexity in the readout scheme of the qubits is obtained.

Additionally, the quantum computational depth of the pUCCD quantum circuit scales linearly with the number of orbitals (MOs) N, a significant improvement over conventional UCCSD circuit depth which is upper-bounded by O( [symbol] ) and which can be reduced by a linear factor using parallelization techniques depending on the qubit lattice connectivity.

In an embodiment, the electron singlet state may be configured to only include molecular orbitals that are occupied or not occupied by electron pairs, preferably a qubit includes a first qubit state |1 [symbol] representing a molecular orbital that [symbol] is occupied with an electron pair and a second qubit state |0 [symbol] representing a molecular orbital that is not occupied with an electron pair.

In an embodiment, the Hamiltonian may describe a quantum chemistry (electronic structure) system that is defined in terms of electron-pair annihilation operators, preferably hard-core bosonic annihilation operators.

In an embodiment, the Hamiltonian may be described based on the following equation:

$${\hat{H}}_{r} = C + {\sum }_{p , q} {h}_{p , q}^{( r 1 )} \text{?} + {\sum }_{p \neq q} {h}_{p , q}^{( r 1 )} \text{?}$$

$$\text{?} \text{indicates text missing or illegible when filed}$$

In an embodiment, the Hamiltonian may describe a quantum chemistry (electronic structure) system that is defined in terms of Pauli spin (qubit) operators.
In an embodiment, the Hamiltonian may be described based on the following equation:

$$\text{?}$$

$$\text{?} \text{indicates text missing or illegible when filed}$$

In an embodiment, the paired-electron unitary coupled cluster with double-excitations (pUCCD) ansatz may be defined in terms of electron-pair annihilation operators, hard-core bosonic annihilation operators.

In an embodiment, the pUCCD ansatz may be defined by the following equation:

$$\text{?}$$

$$\text{?} \text{indicates text missing or illegible when filed}$$

In an embodiment, the paired-electron unitary coupled cluster with double-excitations (pUCCD) ansatz may be defined in terms of Pauli spin (qubit), In an embodiment, the pUCCD ansatz may be defined by the following equation:

$$\text{?}$$

$$\text{?} \text{indicates text missing or illegible when filed}$$

Restricting the subspace to only include superpositions of electron singlet-configurations also allows for Quantum Phase Estimation to be implemented more efficiently than previously possible. In this way, fault-tolerant quantum computational (FTQC) devices can gain a significant advancement in computational accuracy with less quantum resources and time.

The restricted Hamiltonian method can readily be extended to include higher-order excitations as desired for accuracy reasons, interpolating towards fully un-restricted, where the trade-off should be made by including higher-order terms for accuracy at a cost of increased run-time due to circuit depth and additional sets of non-commuting operators in the Hamiltonian.

In an embodiment, the trial state and the energy may be determined based on a variational scheme, preferably a variational quantum eigenvalue (VQE) system.

In an embodiment, the VQE system may include a quantum processor and a classical processor. Further, in an embodiment, the quantum computer may include a quantum state preparation module for preparing a number of qubits in an initial state |Ψ₀ [symbol] and an energy estimation module for estimating the energy by determining expectation values of the energy associated with the initial state. In an embodiment, the determining of the expectation value includes preparing a parameterized quantum state |Ψ(θ) [symbol] on the quantum device by applying a parameterized unitary U(\vec{θ}) to the initial state: U(\vec{θ})|Ψ₀ [symbol] =|Ψ(\vec{θ}) [symbol] . In an embodiment, the parameterized unitary, may be executed as a parametrized quantum circuit which simulates an pUCCD ansatz, wherein θ represents the parameters of the ansatz, e.g. a list of amplitudes

In an embodiment, the quantum state preparation module and energy estimation module may be implemented as a gate-based qubit quantum circuit comprising N qubits which may be configured in an initial state. In an embodiment, the measuring of the energy may include determining the quantum state of the qubits by applying a unitary ansatz to the initial state. In an embodiment, the application of the unitary may include the sequential application of a predetermined number of gate operations. In some embodiments, such gate operations may be defined as a Trotter step. In an embodiment, a basis rotation R_{1-N} may be applied to at least part of the qubits. In an embodiment, qubits may be read out by a readout circuit, for obtaining an expectation values of the Pauli terms <P₁(θ)>, . . . , <P_{N}(θ)>. These expectation values are related to the matrix elements in the qubit Hamiltonian. The expectation values of each of these Pauli terms may be either “0” or “1”, thus representing a string of zero's and one's (a bitstring). In an embodiment, expectation values may be provided by an averaging module that runs on the classical computer. In an embodiment, the averaging module may be configured to determine an expectation value of the energy [symbol] H [symbol] (\vec{θ}). This expectation value may be provided to an optimization algorithm, which may determine a new set of parameters \vec{θ}′ 316 for input to the quantum circuit for a next measurement round in an optimization loop.

In an embodiment, the determining a trial state may include: initializing qubits of the quantum computer based on parameters, preferably coupled cluster amplitudes, which are computed on information of the quantum chemistry system.

In an embodiment, determining a trial state may include: sequentially applying gate operations of the quantum circuit to pairs of qubits, an application of a gate causing the pair of qubits to interact with each other.

In an embodiment, determining a trial state may include: applying a sequence of gate operations to the qubits of the quantum circuit; applying a basis rotation to each qubit; and, performing a qubit readout for each qubit.

In an embodiment, the gate operation may be a singlet-state simulation (SSS) gate operation, including a partial-swap gate operation, partial-swap gate performs an entangling operation which simulates partially distributing an electron pair among two molecular orbitals.

In an embodiment, the SSS gate operation may include a partial-swap gate operation and at least a full-swap gate operation. A full-swap gate swap(i, j) swapping qubit labels in order to bring every qubit which was occupied next to every other qubit which was not occupied. A partial-swap gate operation performing an entangling operation which simulates partially distributing an electron pair among two molecular orbitals. In an embodiment, at least the partial operation may be parametrized by a gate angle θ.

In an embodiment, the gate operation may be a singlet-state simulation (SSS) gate operation, including a full-swap gate, which may swap the logical qubit labels in order to bring every logical qubit which was occupied next to every other logical qubit which was not occupied.

Thus, the pUCCD protocol enables efficient use of the swap network for simulation, such that the qubit lattice connectivity requirement is relaxed considerably as compared to previous UCC proposals. Only a single linear chain needs to be defined across the lattice to reach the proven-to-be-minimal linear gate depth.

The only required unitary quantum operations in pUCCD are single-qubit rotations and the SSS, which is a two-qubit partial-swap operation. As currently all universal gate-based qubit quantum computers are based on decompositions into single- and two-qubit gate operations with high fidelity and good multi-qubit gates are still out-of-reach, the SSS operation can generally be implemented more naturally than a conventional UCCSD cluster exponential consisting of four-body Pauli terms.

In an embodiment, the trial state and the energy may be determined based on a quantum phase estimation (QPE) scheme.

In a further aspect, the invention may relate to a system for simulating a quantum chemistry system comprising: a memory device including computer-executable instructions and a processor connected to the memory device, the processor being configured to perform executable operations comprising: determining a Hamiltonian describing the quantum chemistry system, the Hamiltonian being restricted to electron singlet state configurations; determining a paired-electron unitary coupled cluster with double excitations (pUCCD) ansatz, the ansatz being restricted to electron singlet state configurations; mapping the pUCCD ansatz to qubit operations of the quantum circuit, the quantum circuit comprising a set of qubits and gates for enabling pairs of qubits to interact with each other; determining a trial state on the quantum circuit by applying the qubit operations defined by the mapped restricted pUCCD ansatz to the qubits; and, determining an energy of the quantum chemistry system based on the trial state and the restricted Hamiltonian.

In a further aspect, the invention may relate to a system for simulating a quantum chemistry system, the system comprising: a computer readable storage medium having computer readable program code embodied therewith, and a processor, preferably a microprocessor, coupled to the computer readable storage medium, wherein responsive to executing the computer readable program code, the processor is configured to perform executable operations comprising: configuring a data processing system comprising a classical computer connected to a quantum computer; receiving or determining, by the classical computer, information on a Hamiltonian describing the quantum chemistry system, the Hamiltonian being a hard-core bosonic Hamiltonian, which restricted to electron singlet state configurations; receiving or determining, by the classical computer, information on a paired-electron unitary coupled cluster with double excitations (pUCCD) ansatz, the ansatz being restricted to electron singlet state configurations; transforming, by the classical computer, the pUCCD ansatz into a quantum circuit, the quantum circuit representing a sequence of qubit operations; executing, by the quantum computer, the quantum circuit, the execution including applying the sequence of gate operations to qubits of the quantum computer; receiving, by the classical computer, a trial state, the trial state including measured expectation values of the Hamiltonian; and, determining an energy of the quantum chemistry system based on the trial state.

In an embodiment, the electron singlet state configurations may only include molecular orbitals that are occupied or not occupied by electron pairs, preferably a qubit includes a first qubit state |1 [symbol] representing a molecular orbital that is occupied with an electron pair and a second qubit state |0 [symbol] representing a molecular orbital that is not occupied with an electron pair.

In an embodiment, the trial energy estimation may be corrected using error mitigation through post-selection in the particle basis of the electronic structure Hamiltonian model.

In a further aspect, the invention may also relate to a computer program product comprising one or more computer-readable storage devices, and program instructions stored on at least one of the one or more storage devices, the stored program instructions comprising instructions for: configuring a data processing system comprising a classical computer connected to a quantum computer; receiving or determining, by the classical computer, information on a Hamiltonian describing the quantum chemistry system, the Hamiltonian being a hard-core bosonic Hamiltonian, which restricted to electron singlet state configurations; receiving or determining, by the classical computer, information on a paired-electron unitary coupled cluster with double excitations (pUCCD) ansatz, the ansatz being restricted to electron singlet state configurations; transforming, by the classical computer, the pUCCD ansatz into a quantum circuit, the quantum circuit representing a sequence of qubit operations; executing, by the quantum computer, the quantum circuit, the execution including applying the sequence of gate operations to qubits of the quantum computer; receiving, by the classical computer, a trial state, the trial state including measured expectation values of the Hamiltonian; and, determining an energy of the quantum chemistry system based on the trial state.

Further, the system for simulating a quantum chemistry system and the computer program product may further include executable operations and/or computer instructions to perform any of the steps described above.

The invention may further relate to a non-transitory computer-readable storage medium storing at least one software code portion, the software code portion, when executed or processed by a computer, is configured to perform any of the method steps as described above.

The invention will be further illustrated with reference to the attached drawings, which schematically will show embodiments according to the invention. It will be understood that the invention is not in any way restricted to these specific embodiments.

### BRIEF DESCRIPTION OF THE DRAWINGS

FIG. 1 schematically depicts a system for simulating quantum chemistry using quantum computation according to an embodiment of the invention;

FIG. 2 schematically depicts a flow diagram of a method for simulating quantum chemistry using quantum computation according to an embodiment of the invention;

FIGS. 3A and 3B depict a schematic of a variational quantum eigenvalue solver system according to an embodiment of the invention;

FIG. 4A-4C depict a quantum circuit for efficient simulation of quantum chemistry, using pulses to control quantum hardware components, according to an embodiment of the invention;

FIG. 5 a variational quantum eigensolver VQE scheme for efficient quantum chemistry simulation according to an embodiment of the invention;

FIG. 6 depicts a graph of the groundstate energy of the Lithium-hydride molecule as a function of interatomic spacing;

FIG. 7 depicts the LiH groundstate energy simulation error as a function of interatomic spacing;

FIG. 8 depicts a quantum circuit for efficient simulation of quantum chemistry according to another embodiment of the invention.

FIG. 9A-9C depicts a quantum error mitigation technique specific to this quantum chemistry simulation protocol for mitigating noise/errors on realistic noisy quantum hardware

### DETAILED DESCRIPTION

In this particular application, a molecular chemistry ‘simulation’ may refer to the determination of the electronic ground state configuration energy with respect to the energy of a quantum chemistry system. For example, (at least) two separate atoms, e.g. two Li atoms, being at rest at a large distance away from each other. The accurate determination of such energy profiles as a function of atomic geometry is essential to the field of quantum chemistry. For example, reaction kinetics are highly dependent on Potential Energy Curves (PECs) as a function of geometrical configuration and knowledge of such energy profiles are essential in understanding and improving the reaction. An aspect of the invention aims to simulate such class of problems on a quantum computer in a manner that is polynomially more efficient than with conventional classical approximate methods such as CCSD(T).

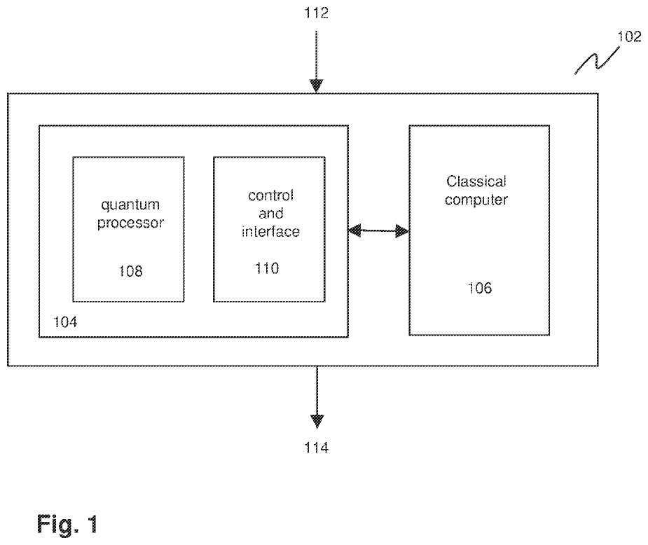

FIG. 1 depicts a system for simulating molecular processes using quantum computation. The simulation system 102 may include a quantum computer system 104 comprising one or more quantum processors 108, e.g. a gate-based qubit quantum processor, and a controller system 110 comprising input output (I/O) devices which form an interface between the quantum processor and the outside world. The quantum computer system may be connected to a classical computer 106 comprising one or more classical processors. For example, the controller system may include a microwave system for generating microwave pulses which are used to manipulate the qubits. Further, the controller may include readout circuitry for readout of the qubits. For at least a part such readout circuitry may be located or integrated with the chip that includes the qubits.

The system may further comprise a (purely classical information) input 112 and an (purely classical information) output 114. Input data may include information about the quantum chemistry system that is needed for the simulation. This information may include the number of electrons, choice of basis set, active and frozen space indices, self-consistent field calculated electron-electron integrals, atomic numbers and geometry, algorithm hyperparameters and optimization settings, etc. The input data may be used by the system to classically calculate values, e.g. amplitudes of excitations, which may be used to initialize a quantum circuit that may be executed on the quantum processor. In particular, the input data may be used by the system to construct quantum circuits, representing a sequence of qubit operations. The quantum circuit may be translated into sequences of pulses, e.g. microwave pulses, which may be used to initialize and control qubit operations according to the quantum circuit. Similarly, output data may include ground state and/or excited state energies of the quantum system, correlator operator expectation values, optimization convergence results, optimized quantum circuit parameters and hyperparameters, and other classical data.

Each of the one or more quantum processors may comprise a set of controllable two-level systems referred to as qubits. The two levels are |0> and |1> and the wave function of a N-qubit quantum processor may be regarded as a superposition of 2^{N} of these basis states. Examples of such quantum processors include noisy intermediate-scale quantum (NISQ) computing devices and fault tolerant quantum computing (FTQC) devices.

The quantum processor may be configured to execute quantum algorithms in accordance with the qubits operations of a quantum circuit. For example, a quantum circuit maybe used to encode a Hamiltonian describing a quantum system, e.g. a molecular chemical system, on the set of N qubits. The quantum processor may be implemented as a gate-based qubit quantum device, which allows initialization of the qubits into an initial state, interactions between the qubits by sequentially applying quantum gates between different qubits and subsequent measurement of the qubits' states. To that end, the input devices may be configured to configure the quantum processor in an initial state and to control gates that realize interactions between the qubits. Similarly, the output devices may include readout circuitry for readout of the qubits which may be used to determine a measure of the energy associated with the expectation value of the Hamiltonian of the system taken over the prepared state.

The Hamiltonian describing a chemical system may comprise several parts. In the so-called Born-Oppenheimer approximation, molecular nuclei may be regarded as stationary on the timescales involved with electron dynamics. In that case simulating the Hamiltonian is the most relevant challenge to solve. In second quantization, the electronic many-body Hamiltonian of the chemical system may be written as follows (equation 1):

$$\text{?}$$

$$\text{?} \text{indicates text missing or illegible when filed}$$

where C is a constant offset, and {h,p,q,s} are fermionic mode indices, a, is the fermionic annihilation operator of the p-th fermionic mode and h_{p,q} and h_{p,q,r,s} are Hamiltonian matrix elements which are integrals over the electron-electron electric field interactions. These integrals are classically computationally tractable and computable using self-consistent-field methods. The number of terms in this Hamiltonian is [symbol] (N⁴).

The number of fermionic modes and the values of the matrix elements also depend on the choice of a basis set, i.e. an orthogonal set of quantum wavefunctions decomposing the overall state of the many-body electronic system. These wavefunctions may indeed be decomposed as a sum of any complete orthogonal set of eigenfunctions. For example, in the STO-6G basis, six Molecular Orbitals (MOs) are used to describe the lithium-hydride system. As electrons are spin-1/2 particles, two of them can occupy a single fermionic mode (spin-up and spin-down). This brings the total number of spin-orbitals (SOs) to twelve. The fermionic Hamiltonian of equation (1) may be mapped to qubits of a gate-based quantum computer using a variety of transformations, for example the Jordan-Wigner transformation, resulting in long Pauli-string operators for every fermionic term which may have the form of \hat{α}ₚ^{†}\hat{α}_{q}^{†}\hat{α}ᵣ\hat{α}ₛ.

In an embodiment, the qubit requirement for a given number of spin-orbitals may be reduced by using an approximate mapping transformation which hereafter may be referred to as the electron singlet-state approximation. In this approximation, it is assumed that in systems with not so many higher-order electron correlations, electrons occupying certain molecular orbitals always stay in, and move as, pairs. This approximation means restricting the Hilbert space from all possible many-body electronic states to only those where molecular orbitals are only occupied by electron pairs (either there are no electrons, or exactly two, or a superposition of those two states, but no single-electron occupation).

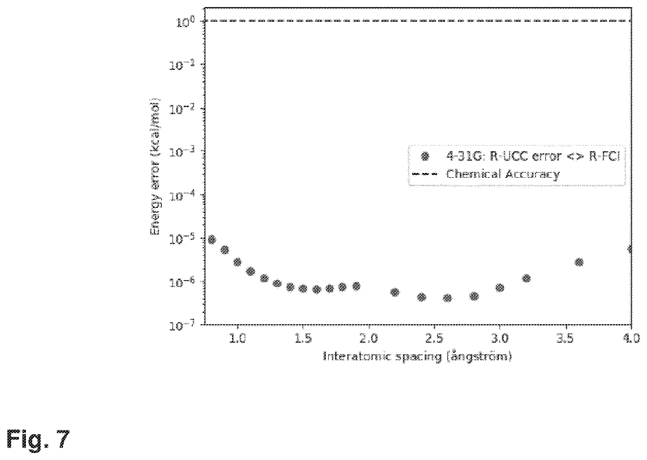

The electron singlet approximation allows to map a problem of a given problem size to half as many qubits. FIG. 7 depicts a table of number of basis functions and number of qubits required to map the basis functions for a UCCSD scheme and a pUCCD scheme. As shown in the table, the electron singlet approximation allows to map the six basis functions in the STO-6G basis set for LiH to six qubits instead of the conventional twelve qubits. Alternatively, the same qubit number may be used with a larger basis set and assign just one qubit to each basis function instead of two. For example, in the 431-G basis set, 11 molecular orbitals result in 22 fermionic orbitals, which can be mapped to 11 qubits in the electron singlet approximation. In this way, with 12 qubits as a quantum resource, one may either simulate LiH in unrestricted STO-6G or in restricted 4-31G.

Each logical qubit has two internal states, 0 and 1. In the electron singlet-state approximation the absence or presence of a pair of electrons in that orbital may be represented by these two qubit states, respectively. A pair of electrons occupying a single molecular orbital is always composed of a spin up and spin down electron (because of Pauli's exclusion principle), therefore the net spin equals zero. In that case, the entire system may be described as a set of N two-level bosonic systems, meaning each bosonic mode has a maximum occupancy of one, associated with each molecular orbital (MO). This is known as a hard-core boson (HCB) system. In that case the restricted Hamiltonian Ĥᵣ, may be written as (equation 2):

$$\text{?}$$

$$\text{?} \text{indicates text missing or illegible when filed}$$

wherein {h,p,q,s} are MO indices, \hat{b}ₚ, is the annihilation operator of the p-th hard-core bosonic mode with anti-commutator {\hat{b}ₚ, \hat{b}_{q}^{†}}=δ_{p,q}, and commutator [\hat{b}ₚ, b_{q}]=[\hat{b}ₚ^{†}, \hat{b}_{q}^{†}]=0 and h_{p,q}^{(r1)} and h_{p,q}^{(r2)} are Hamiltonian matrix elements which are integrals over the energy contributions. In this restricted case, the total number of terms in the Hamiltonian scales as N² (as compared to N⁴ in the unrestricted Hamiltonian).

The restricted Hamiltonian in the electron singlet-state approximation (as represented by equation 2) can be mapped to Pauli spin operators describing the qubit dynamics of a gate-based quantum simulator using the following transformation rule (equation 3):

$$\text{?}$$

$$\text{?} \text{indicates text missing or illegible when filed}$$

where \hat{σ}ₚ^{X} and \hat{σ}ₚ^{Y}, are the pauli spin x- and y-qubit operators respectively. Due to hermiticity of equation 2, some terms vanish, and the full Hamiltonian—referred to as the qubit Hamiltonian Ĥ_{qb}— may be given by the following equation 4:

$$\text{?}$$

$$\text{?} \text{indicates text missing or illegible when filed}$$

wherein \hat{σ}ₚ^{Z} is the Pauli spin z-qubit operator and f, the unity operator.

From the above equation, it is clear that there are three groups that can be identified as commuting with each other: all terms involving Z and ZZ, all terms involving XX, and all terms involving YY. The terms within each of these groups of operators can be measured simultaneously, which means the total N² terms can actually be simulated in just three steps (first Z/ZZ, then XX, then YY) and classical post-processing can tabulate and sum these results. In order to measure the terms based on Z or ZZ, the qubits are simply measured directly in the original basis. In that case, no rotation before measurement is needed because, the Z basis is already the natural basis to measure in for qubit-based quantum hardware, by definition. Hence, beside the gain in runtime, a significant reduction in the complexity in the readout scheme of the qubits is provided.

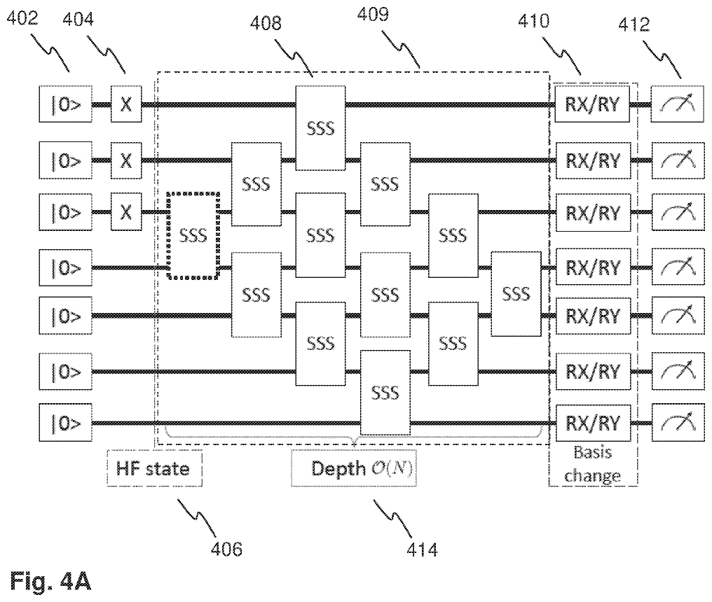

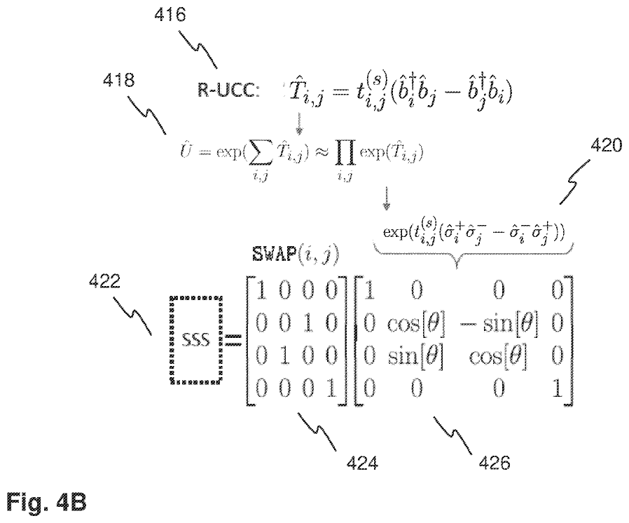

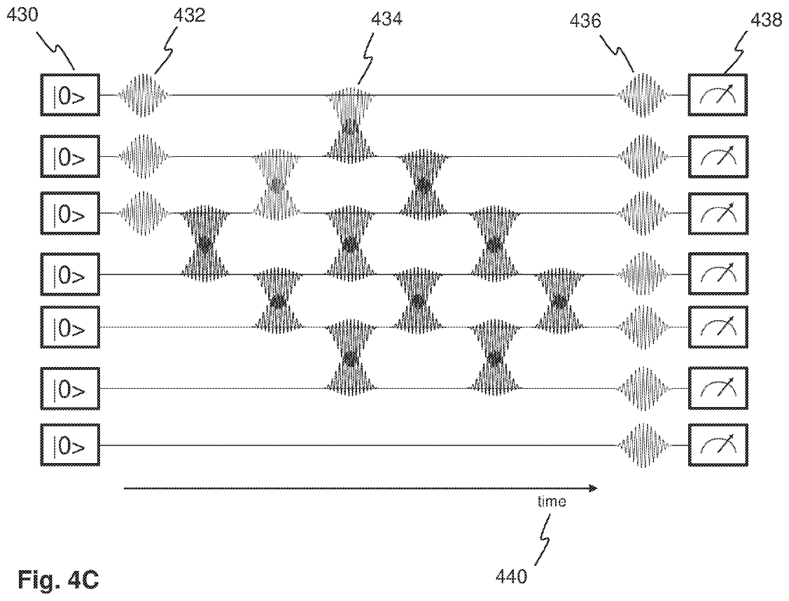

In order to measure the XX terms, one would rotate all qubits by 90° from X basis to Z basis, using an RY rotation (410 in FIG. 4A). Conversely, to measure all YY terms, one would rotate all qubits by 90° from Y basis to Z basis, using an RX rotation (410 in FIG. 4A). After rotating, a Z-basis measurement would give the effective result in the corresponding X or Y basis instead, which can then be post-processed to estimate the expectation value of all terms in the Hamiltonian. This allows for measuring in three separate groups all N² terms.

The restricted Hamiltonian, a hard-core bosonic Hamiltonian, therefore hardware efficient scheme for executing a quantum algorithm for modelling quantum chemistry. It provides a reduction in scaling which is in stark contrast to the original fermionic Hamiltonian mapping to qubits, which even after grouping, results in N⁴ mutually-commuting terms. This is because a fermionic Hamiltonian cannot be mapped to qubits directly/naturally, whereas the hard-core bosonic Hamiltonian is directly supported on qubits due to the matching commutation relations of the shared SU(2) group. Fermionic Hamiltonians can be mapped to qubits using for example the Jordan-Wigner transformation. This transformation maps each fermionic operator to a string of Pauli operators (or Pauli ‘words’), with terms like X/Y/Z spread around, which support on several qubits in a chain potentially covering the whole quantum processor. This introduces huge complexity in the measurement scheme, as each qubit would need to be rotated to a particular basis and the terms cannot be grouped into a lower number of shared tensor-product bases than O(N⁴). Furthermore, the shot-noise error is worse for longer Pauli words (worse for greater non-locality of the Hamiltonian).

As a particular numerical example of the difference in runtime scaling between N⁴ and 3, an example of simulating ABP in an accurate basis set may be regarded; In the year 2020, the energy of 2-aminobenzophenone (ABP, C13H11NO) was computed in the large def2-QZVPPD basis set using a CCSD(T) approach. That calculation, utilizing the density-fitting approximation, correlated 90 non-FC electrons among 1569 orbitals and was completed on 224 classical computer cores in 68 hours. This sets a rough limit of what is still feasible on a classical supercomputer now. On a conventional UCCSD approach, this large number of orbitals would mean repeating the quantum circuit estimations at least N⁴˜10¹⁴ times the number of shots per term, for each VQE iteration, while in the method presented here, this would be three times the number of shots per term, resulting in a wall-clock runtime improvement of 10¹⁴. This extra time can then instead be used to consider a larger basis set, larger problem, or running more calculations per unit of time.

A first step of any quantum simulation algorithm is the preparation of a trial state. The success of an algorithm for determining eigenenergies of the Hamiltonian depends on the quality of the state preparation and its closeness to the actual eigenstate of interest. A good initial guess for the groundstate of the restricted Hamiltonian Ĥᵣ is the Hartree-Fock (HF) state, which in this case is just a product state with the nₑ lowest-energy MOs occupied with a single pair of electrons. For systems with even number of electrons, the HF state in the singlet-subspace approximation describes the same state as without the approximation, and the groundstate energy expectation value of this state over the restricted Hamiltonian is also equal to the groundstate energy of the full Hamiltonian.

Such a simple guess state often does not capture the complexity of an entangled ground state. Therefore, after preparing the HF state on a qubit lattice, i.e. a collection, e.g. a grid or a line, of hardware qubits with a certain connectivity amongst them, an additional circuit may be applied to the array which prepares the trial state, i.e. a more general ansatz including higher-order correlators. In principle, an eigenstate of the many-body Hamiltonian could contain many complicated correlations, which implies that a large number of entangling operations need to be applied to the HF state (which is a product state) in order to produce this highly entangled state.

In practice however, single/double/triple single-particle excitations are often enough to bring the HF state very close to the groundstate. A scheme grounded in this idea, is known as the coupled-cluster method, taking for example single/double(triple) (CCSD(T)) excitations. In classical computational chemistry, the non-unitary form of this operation is applied to the HF state as an approximation, because the unitary matrix exponential is very costly to compute. However, on a specific quantum simulator device the unitary evolution over a coupled-cluster operator is naturally implementable.

In conventional UCCSD methods, unitary operator 0 may be constructed based on the single-particle coupled-cluster scheme according to the following equations 5-8:

$$\text{?}$$

$$\text{?} \text{indicates text missing or illegible when filed}$$

wherein, \hat{T}_{X} \hat{\hat{T}}_{2,1} and \hat{T}_{2,2} represent CC operators with excitations involving two orbitals and a single electron, excitations involving two distinct orbitals and two electrons, and excitations involving up to four distinct orbitals and two electrons, respectively. \hat{α}ᵢ is the fermionic annihilation operator of the i-th fermionic mode and tᵢⱼ⁽¹⁾, tᵢⱼ^{(2,1)} and tᵢⱼ^{2,2} represent individual excitation amplitudes for each overall operator \hat{T}₁ \hat{T}_{2.1} and \hat{T}_{2,2} respectively. To simulate this as a quantum circuit, one may Trotterize the evolution and apply each term sequentially as e.g. described in the article by M. Suzuki, Fractal decomposition of exponential operators with applications to many-body theories and Monte Carlo simulations, Physics Letters A 146, 319 (1990). If the amplitudes are not too big, this Trotterization leads only to small errors with the true UCCSD state.

Within the electron-singlet approximation however, the above-mentioned UCC ansatz changes to only include pairs of electrons being removed from an occupied MO and moved to a virtual MO. Therefore, the following approximate ansatz unitary, for reaching an approximate groundstate to the problem Hamiltonian may be used (equations 9-11) where the superscript (s) denotes the electron-singlet approximation:

$$\text{?}$$

$$\text{?} \text{indicates text missing or illegible when filed}$$

Here, \hat{T}ₛ represents the singlet-restricted coupled cluster operator, tᵢⱼ^{(s)}) represents the amplitudes associated with each coupled cluster term, tᵢⱼ^{(s)}) is the corresponding term indexed on the qubit lattice instead, \hat{σ}ₚ^{X} and \hat{σ}ₚ^{Y} are the pauli spin x- and y-qubit operators respectively, and \hat{σ}ₚ⁺ and \hat{σ}ₚ⁻ are the pauli spin raising and lowering operators respectively. From the form of the ansatz it is clear the unitary operation is particle and spin conserving. This ansatz may henceforth be referred to as the paired-electron unitary coupled cluster with double excitations (pUCCD) ansatz. \hat{T}ₛ can be mapped to Pauli spin operators describing the qubit dynamics of a gate-based quantum simulator using the transformation rule of the above described equation 3. This mapping results in qubit operator \hat{T}_{qb} (equation 11) which expresses the pUCCD ansatz in terms of Pauli-spin operators of a gate-based qubit-based quantum circuit.

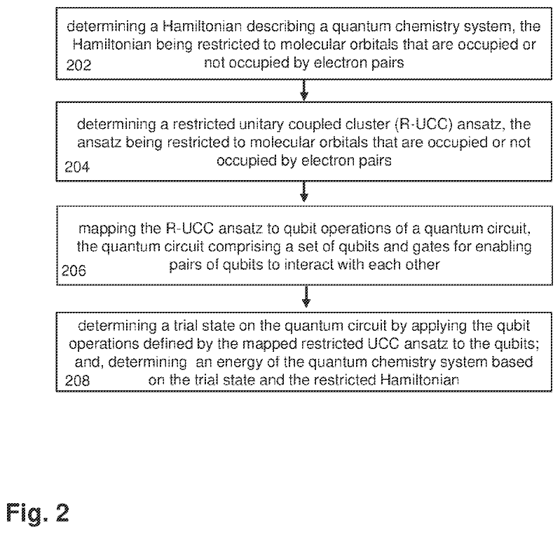

Thus, based on the above-described scheme efficient simulation of quantum chemistry systems can be achieved. FIG. 2 depicts a schematic of such simulation process which includes determining a Hamiltonian describing the quantum chemistry system, the Hamiltonian being restricted to electron singlet state configurations (step 202). Further, a paired-electron unitary coupled cluster with double excitations (pUCCD) ansatz may be determined wherein, the ansatz is restricted to electron singlet state configurations (step 204). The pUCCD ansatz may then be mapped to qubit operations of a quantum circuit, the quantum circuit comprising a set of qubits and gates for enabling pairs of qubits to interact with each other (e.g. enable entanglement between the qubits (step 206). A trial state may be determined on the quantum circuit by applying the qubit operations defined by the restricted UCC ansatz mapped to the qubits; and, determining an energy of the system based on the trial state and the restricted Hamiltonian (step 208).

The Trotterized version of \hat{T}_{qb} can be described as a parametric circuit for a gate-based quantum computer. After performing a trial state preparation with such a parametrized circuit, the energy may be calculated in different ways. In an embodiment, a Quantum Phase Estimation may be which would yield arbitrary precision but has coherence requirements that are too stringent for current-era NISQ hardware. In another embodiment, Hamiltonian averaging and variationally optimizing over the resulting energy expectation value may be used. Such method may be referred to as a variational quantum eigensolver (VQE) scheme.

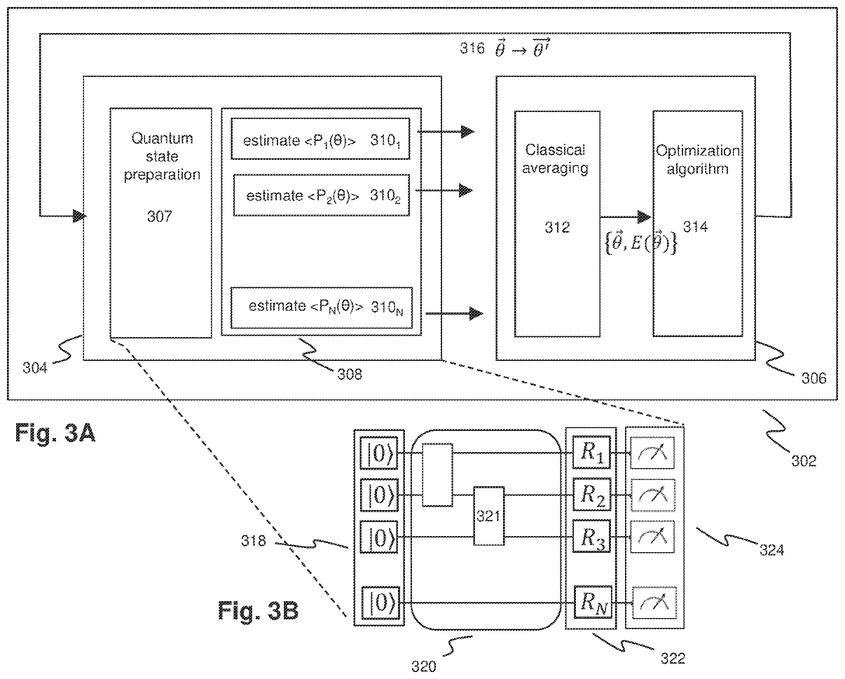

FIGS. 3A and 3B depict a schematic of a variational quantum eigensolver VQE system that may be used for quantum chemistry simulation using the above-described paired-electron unitary coupled cluster with double excitations (pUCCD) ansatz. As shown in FIG. 3A, the VQE system 302 may include a quantum processor 304 and a classical processor 306. As shown in FIG. 3B the quantum computer may include a quantum state preparation module 307 for preparing a number of qubits in an initial state |Ψ₀ [symbol] and an energy estimation module 308 for estimating the energy by determining expectation values 310₁₋ₘ of the energy associated with the initial state. The determining of the expectation value includes preparing a parameterized quantum state |Ψ\vec{θ} [symbol] on the quantum device by applying a parameterized unitary U(\vec{θ}) to the initial state: U(\vec{θ})|Ψ₀ [symbol] =|Ψ\vec{θ} [symbol] . Here the initial state is relatively straightforward to prepare and the parameterize unitary, in this case specifically, is executed as a parametrized circuit which simulates the above-described pUCCD ansatz. θ represents the parameters of the ansatz, which in this case consists of the list of amplitudes

As shown in FIG. 3B, the quantum state preparation module and energy estimation module may be implemented as a gate-based qubit quantum circuit comprising N qubits 318 which may be configured in an initial state. The measuring of the energy may include determining the quantum state of the system by applying a unitary ansatz to the initial state. The application of the unitary includes the sequential application of a predetermined number of quantum gates 321, which may be referred to as a Trotter step, and subsequently applying a basis rotation R_{1-N} 322 to each qubit. Each qubit may be read out by a readout circuit 324, which aims to estimate the expectation values of the Pauli terms <P₁(\vec{θ})>, . . . , <P_{N}(\vec{θ})> which are given by the matrix elements in the qubit Hamiltonian of Equation 4. The expectation values of each of these Pauli terms may be either “0” or “1”, thus representing a string of zero's and one's, i.e. a bitstring. These values may be provided by an averaging module 312 that runs on the classical computer. The averaging module may be configured to determine an expectation value of the energy [symbol] H [symbol] (\vec{θ}), which is represented by a real number. This value may be provided to an optimization algorithm 314, which may produce a new set of parameters \vec{θ} 316 for input to the quantum circuit for a next measurement round.

FIGS. 4A and 4B depict a quantum circuit for efficient simulation of quantum chemistry according to an embodiment of the invention. In particular, FIG. 4A depicts a more detailed illustration of a quantum hardware device that is capable of approximately simulating the above-described unitary ansatz using a so-called Trotter step consisting of a set of sequential and parallel gate operations. In particular, this figure illustrates how a Trotter step can be simulated on a quantum hardware device using a discrete set of pre-programmed unitary operations (or ‘gates’), which are variationally optimized based on a VQE scheme as described with reference to FIGS. 3A and 3B to yield an approximate groundstate energy. The circuit has a linear circuit depth, i.e. the maximum number of sequential gate operations scales linearly with the number of qubits N. The figure shows a quantum hardware circuit wherein a number of qubits 402, in this example seven qubits, are initialized in the |0 [symbol] state. In the quantum circuit each qubit represents (simulates) an electron pair, wherein the |1 [symbol] state represents a molecular orbital that is occupied with an electron pair and the |0 [symbol] state represents a molecular orbital that is not occupied with an electron pair.

The lowest nₑ molecular orbitals, in this example three orbitals, may be populated with electron-pairs using a so-called X-gate (or ‘NOT-gate’) 404, switching the |0 [symbol] state of the first three MO to the occupied |1 [symbol] state. After application of the X-gate, the qubits may be configured in a Hartee-Fock (HF) initial state |Ψ₀ [symbol] 406. Further, at the start of the process, the parameters of the quantum circuit, e.g. the coupled cluster amplitudes tᵢⱼ^{(s)} may be initialized based on estimates which may be computed based on a classical model. These estimates may be computed based on information on the chemistry problem. This information may be provided as input data to the system. The quantum circuit representing the paired-electron unitary coupled cluster with double excitations (pUCCD) ansatz may then be executed by sequentially applying predetermined gate operations to the qubits. The gate operations 408 are applied in a so-called parallelized swap network 409, followed by basis rotations 410 depending on the particular qubit Hamiltonian element to be estimated, and followed by performing qubit readout 412.

FIG. 4B depicts a gate operation according to an embodiment of the invention. In particular, this figure depicts a gate operation which may be referred to as a singlet-state simulation (SSS) gate operation 422. Individual pUCCD terms 416 of the unitary operator Û 418 include swaps between modes (i.e., logical qubit swapping representing swapping pairs of electrons between molecular orbitals). In an embodiment, the full unitary operator 0 may be approximated by a single Trotter step. This is equivalent to approximating the exponential of the sum in 418 with the product of the exponential, which is executed as a sequence of SSS-gates. In another embodiment, the full unitary operator may be approximated by multiple Trotter steps for increased accuracy. As described hereunder in greater detail, a single Trotter step may already show excellent results in UCCSD-VQE experiments.

A SSS-gate 422 may include a partial-swap gate 426 followed by a full-swap gate 424. The full-swap gate swap(i, j) swaps the logical qubit labels in order to bring every logical qubit which was occupied next to every other logical qubit which was not occupied. The partial-swap gate performs an entangling operation which simulates partially distributing an electron pair among two molecular orbitals, parametrized by the gate angle θ. This gate angle θ may be used as an optimization parameter in an optimization loop. In this way, excitations from occupied orbitals to every virtual orbital may be simulated, in a minimal gate-depth of Neven on linear chain of qubits (nearest-neighbor connectivity). The SSS-gate implements the required 2-body terms, which is a more natural implementation on current-day universal gate-based quantum computers than conventional UCCSD cluster exponential four-body terms.

In the measurement phase, one may only measure sets of non-commuting operators due to the fundamental limits imposed on measurement by the laws of quantum mechanics. In conventional UCCSD-VQE, this set size scales in size as [symbol] (N⁴), increasing the overall computation time this factor, and the theoretical upper bound on total measurements is [symbol] (N⁶) may be brought down to an upper bound of O(N⁶) measurements for most realistic molecules in a Gaussian type orbital basis set as e.g. described in the article by McClean et al, Exploiting Locality in Quantum Computation for Quantum Chemistry, The Journal of Physical Chemistry Letters 5 (24), 4368-4380 2014 (in practice, due to its heuristic nature the scaling could be several orders lower).

However, as described above, in the restricted singlet subspace Hamiltonian method described in this application, all Hamiltonian operators may be sorted into just three groups of mutually commuting sets of operators, i.e.: X-basis, Y-basis and Z-basis parts of the qubit Hamiltonian represented by equation 4. This only provides a constant overhead, [symbol] (1), in the overall complexity due to the measurement phase contribution, with a worst-case-scenario scaling upper bound of [symbol] (N⁴) measurements due to shot repetition requirements at fixed desired accuracy. This is due to the fact that estimation of the energy is performed by repeated bitstring measurements over the qubit lattice wave-function in order to essentially perform tomography, which has a certain shot repetition requirement to be accurate enough (However, this is only theoretically upper bounded, not lower bounded) The final total black-box evaluation time scaling then shows a significant polynomial speedup over the state-of-the-art quantum simulation protocol UCCSD-VQE.

FIG. 4C depicts the same quantum circuit as described with reference to FIG. 4A. In this case however, the figure illustrates the actual physical quantum hardware operations that are executed, plotted versus time 440 on the horizontal axis, gate operations 432, e.g. in the form of microwave pulses of a predetermined frequency, amplitudes and phase for initializing of a part of the qubits 430 of the quantum processor in the |0 [symbol] state; gate operations representing the paired-electron unitary coupled cluster with double excitations (pUCCD) ansatz; gate operations representing basis rotations and readout operations for qubit readout 438. Comparing this diagram to FIG. 4A shows a clear correspondence between the actual physical operation in terms of and its effect in the logical space that this operation will have in terms of the quantum information contained in the qubits which are subsequently read-out.

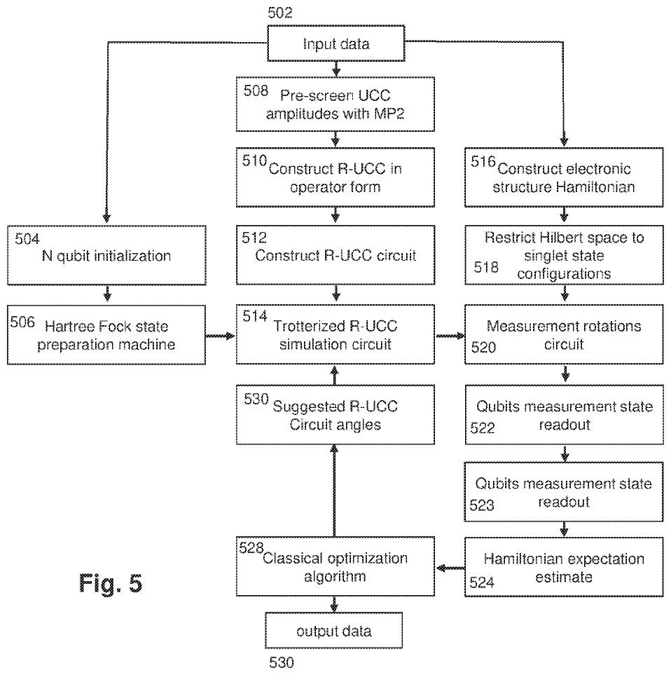

FIG. 5 depicts a workflow for a variational quantum eigensolver VQE scheme for efficient quantum chemistry simulation according to an embodiment of the invention. As shown in the figure, the method may start with providing input data 502 to a VQE system. An example of such VQE system is described above with reference to FIGS. 3 and 4. Here input data may include pre-calculated data known about the molecule. Based on the input data, the VQE system may generate ‘output data’ 530 which may include a simulated Hamiltonian spectrum. The input data may be used to prepare an array of qubits 504 into an initial state 506, i.e. an approximate to the ground state, such as a HF state. The input data may also be used to pre-process certain pUCCD initial values, once. For example, the input data may be used to pre-screen pUCCD amplitudes using Moller-Plesset 2^{nd} order (MP2) perturbation theory 508 and filter out those which are irrelevant, in order to reduce circuit size later on.

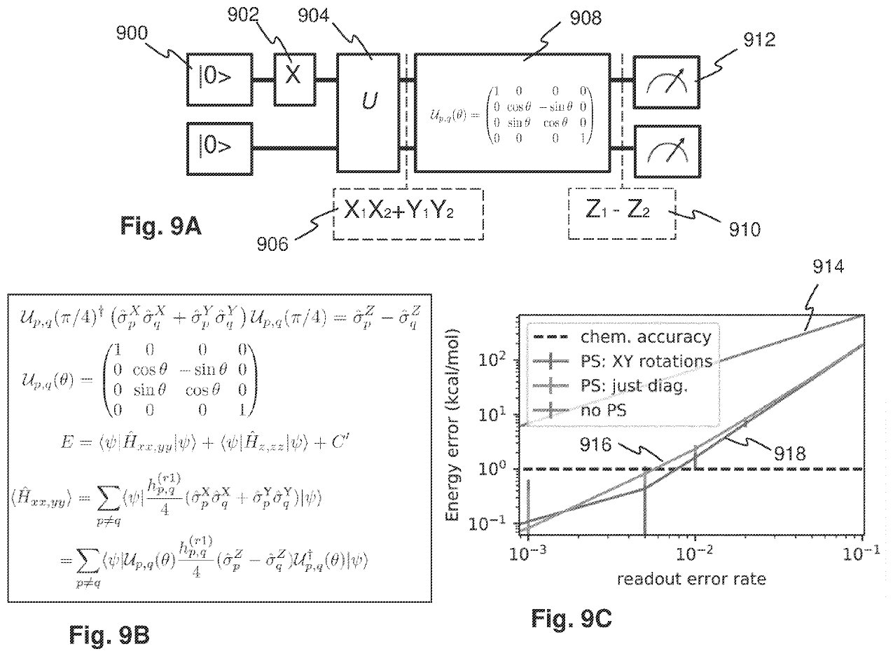

The complete operator form of pUCCD may be constructed 510 which is then converted to a quantum circuit via Suzuki-Trotter decomposition 512. This quantum circuit is then used as the main circuit ansatz applied to the qubits 514. This part is described in more detail with reference to FIGS. 3 and 4. Meanwhile, the input data is also used to construct the electronic structure Hamiltonian 516 (equation 2) which is then restricted to only singlet state configurations 518 (equation 4). The Hamiltonian terms then determine which single-qubit rotations 520 RX or RY are applied to the qubit lattice after the ansatz execution, in order to rotate the basis frame of reference. This is done because the qubits are always measured in the Z-basis in 522. The state of a measured qubit may be “0” or “1”. The collection of measured states may form a ‘bitstring’, which is collected and processed using a classical computer in 523, which is described in detail in FIG. 9, post-selecting the measurement results for error-mitigation purposes. The output of Hamiltonian expectation estimate 524 may be a value, e.g. a real number, representing the energy expectation value, which is which is then fed into a classical optimization routine 528 which may determine new circuit parameters, in particular values for optimization parameter 6, to use 530 for the next iteration of the pUCCD circuit. These new parameter values are the input to the ansatz part of the circuit execution in subsequent rounds, after the N-qubit array has been reset and initialized into the HF-state. The classical optimization algorithm 528 is repeated until convergence, after which the convergence data and final optimized groundstate energy is output 530.

Hereunder an example of a problem mapping in quantum chemistry calculations is described based the above-described the above-described paired-electron unitary coupled cluster with double excitations (pUCCD) ansatz. For this purpose, a simple molecule, the lithium-hydride molecule is used, which is one of the smallest di-atomic molecules with more than two electrons and which is produced by treating lithium metal with hydrogen gas at high temperatures. Its potential applications include hydrogen storage, as a precursor to complex metal hydrides, for shielding or moderation of nuclear reactors. The lithium-hydride molecule may be readily studied with exact methods in computational methods. The example is merely used to illustrate the advantages of the invention and is in no way limiting the gist of the invention in the sense that the invention may be used for efficient quantum simulation of more complex molecules.

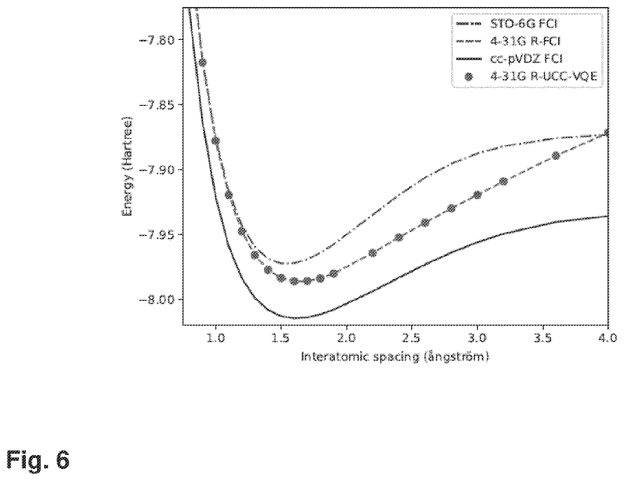

FIG. 6 depicts groundstate energy simulation results for the lithium-hydride molecule as a function of the interatomic spacing. Using the pUCCD-VQE method described above, the energy is computed by Hamiltonian averaging (in this example simulation access to the full wavefunction was available and therefore the result is not stochastic, whereas in an actual experiment it would be). This energy is then variationally optimized and a final groundstate energy estimate was provided. This energy is then compared with the classical computational chemistry methods HF, CCSD and the Exact GS by diagonalization.

Simulations of the pUCCD yield good correspondence to the exact groundstate in the same basis set, over the whole range of interatomic spacings. The results are compared against a much bigger and more accurate basis set, representing an unrestricted diagonalization of the cc-pVDZ set. As shown in FIG. 6, the exact groundstate for the pUCCD ansatz using the 4-31G basis set is much closer to cc-pVDZ results than the unrestricted FCI ansatz using the STO-6G basis set. Hence, results using the pUCCD are more accurate even though the unrestricted STO-6G scheme would be simulated on a comparable number of qubits (12 versus 11 qubits, respectively). The pUCCD method converges closely to the exact diagonalization in that restricted 4-31G basis, while both are better than the best possible result (as UCCSD would be equal to or worse than FCI, in that basis set). Also, taking advantage of the superior efficient mapping of pUCCD, the circuit depth and measurement phase are both polynomially shortened. Thus, when comparing to known unrestricted methods, the pUCCD method allows an accuracy increase while using the same or even less quantum resources.

FIG. 7 depicts the accuracy of pUCCD-VQE compared to the exact diagonalization in the restricted subspace (or ‘R-FCI’) for the 4-31G basis set in LiH. As shown in the figure, the pUCCD-VQE converges variationally to the exact groundstate energy over the whole range of inspected inter-atomic spacing. The energy error is mostly limited to the convergence criterion, and is over 5 orders of magnitude more accurate than chemical accuracy, the gold standard of quantum computational chemistry.

A similar argument can be made in general for the realization of a better computational scaling of quantum algorithms using a restricted-Hilbert space approach within quantum chemistry simulations. Generally, the Hamiltonian described by equation 2 describes only first-order interactions and requires less gate-depth to simulate to arbitrary precision using QPE and similar FTQC algorithms, than does the conventional higher-order Hamiltonian of equation 1. This is because the number of terms considered in the restricted subspace is now [symbol] (N²) instead of [symbol] (N⁴).

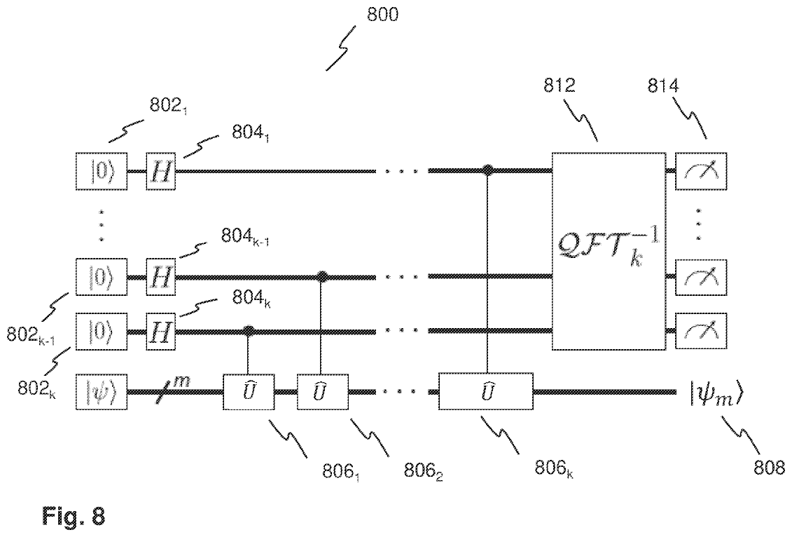

FIG. 8 depicts a quantum circuit for efficient simulation of quantum chemistry according to another embodiment of the invention. In particular, this figure depicts a gate-based quantum circuit 800 for determining the energy based on a Quantum Phase Estimation (QPE) scheme. As shown in this figure, the circuit may comprise a state preparation register 808_{1,2} for preparing a trial state and an ancillary register comprising k qubits 802₁₋ₖ. The qubits of the ancillary register may be prepared in a uniform superposition of all 2^{k} bitstrings. This superposition may be realized using Hadamard gates H 806₁₋ₖ. Then, a sequence of controlled-unitary operations Û 808₁₋ₖ based on the restricted Hamiltonian may be applied to enable interaction between a qubit in the ancillary register and the state preparation register. Application of this unitary operations correspond to Hamiltonian evolution E^{-iHt}. To that end, the quantum circuit is first prepared in a suitable approximate groundstate |Ψ [symbol] . After applying the quantum version of an inverse Fourier transform 810 to the k-qubit register, the qubit registers are readout and a k-bit representation of the eigenenergy of the Hamiltonian may be extracted to an accuracy ε. The groundstate energy can then be found in time proportional to 1/ [symbol] Vₐ|V [symbol] ² and 1/ε, where [symbol] Vₐ|V [symbol] is the overlap between the true groundstate and the approximate groundstate prepared on the state preparation register.

For some systems, it has been proven that the HF-state, a simple product state preparable in [symbol] (1) gates, may have exponentially vanishing overlap with the true groundstate. In that case, a state preparation scheme such as pUCCD is required, or adiabatic state preparation. In either of these cases, substantial benefits in terms of scaling can be achieved, because pUCCD state preparation has only [symbol] (N) depth and adiabatic state preparation at most [symbol] (N²) assuming parallelizable gate operations.

The pUCCD ansatz may be used for other phase estimation part of QPE, the controlled-unitaries describe Hamiltonian evolution and as such require a gate depth scaling at most [symbol] (N³) ( [symbol] terms requiring at most N operations per term) per Trotter step, much less than in the case of a fully unrestricted Gaussian basis set at [symbol] (N⁵) ( [symbol] terms requiring at most N operations per term) per Trotter step, which gives a favorable scaling to the overall computational runtime. The controlled-unitary gate operations are often challenging to realize practically on a quantum device, as it may involve multi-qubit interactions which are hard to implement coherently. In the un-restricted Hamiltonian QPE simulation, these controlled-unitaries involve at most 5-qubit interactions whereas in the restricted Hamiltonian QPE the controlled-unitary operations can be performed with 3-qubit interactions.

The performance enhancement is likewise expected for Kitaev's PEA and Iterative Phase Estimation methods, as the state preparation and controlled-unitary operations remain the main components contributing to the total runtime of the algorithms.

FIG. 9A schematically an NISQ hardware compatible error-mitigation quantum circuit according to an embodiment of the invention. As shown in the figure, the quantum circuit only has O(1) depth two-qubit operations (replacing the O(1) depth single-qubit operations) to measure all terms in the diagonal basis. The quantum circuit is based on the following principles: when estimating the qubit Hamiltonian expectation in the presence of noise on a quantum processor unit (QPU), some of the physical symmetries of the chemistry system that is simulated may be exploited in order to mitigate the induced errors. In particular, the initial reference state, the restricted Hartree-Fock state, has a particular number of excitations (pairs of electrons). Next, the unitary ansatz circuit pUCCD is particle-conserving, which means that the number of electron pairs is conserved. The trial state may be a superposition of basis states with that number of excitations, resulting in only bitstrings with that same number of 1's, in the measurement results. However, if errors occur in the circuit or measurement phase, this may no longer always be the case. As any physical state must conform to the particle-number conservation, the measurement results can be post selected on that condition. This may significantly improve the result; however, there are some caveats. For one, the number of particles being correct does not mean no errors occurred in that case. Also, the above only holds true when measuring in the particle-basis, i.e. the Z operators in the qubit-Hamiltonian.

In FIG. 9B, the mathematics behind this error mitigation scheme is shown. The qubit array may be rotated to other bases than X, Y or Z. In particular, parts of the Hamiltonian acting on pairs of qubits (p,q) could be diagonalized as

U_{p,q}(x/4)¹(σₚ^{X}σ_{q}^{X}+σₚ^{Y}σ_{q}^{Y})U_{p,q}(x/4)=σₚ^{Z}−σ_{q}^{Z}

$$\text{?} = ( \begin{matrix}1 & 0 & 0 & 0 \\ 0 & cos θ & - sin θ & 0 \\ 0 & sin θ & cos θ & 0 \\ 0 & 0 & 0 & 1\end{matrix} )$$

$$\text{?} \text{indicates text missing or illegible when filed}$$

is a Givens rotation over angle θ between qubits p and q, with matrix basis {00\>,|01>,|10>,|11>}. Or, inversely, the system wavefunction could be rotated, first prepared with the regular initialization 900, HF state preparation 902, and variational ansatz 904, before measurement by a circuit-synthesized unitary U^{†} (908) and then the diagonal Zᵢ and Zⱼ operators can be measured simultaneously 910, 912. Hereunder, it is shown how this helps in measuring the expectation value of the chemistry Hamiltonian in qubit form. The task is divided into measuring the expectation value of the XX+YY terms 906, the Z and ZZ terms, and the constant term C′ (which now absorbs the identity terms in addition to the C constant from the original qubit Hamiltonian):

E= [symbol] Ψ|Ĥ_{xx,yy}|Ψ [symbol] + [symbol] Ψ|Ĥ_{z,zz}|Ψ [symbol] +C′

where the term for H_z,zz i may be efficiently evaluated in one go, as the Hamiltonian is diagonal in the qubit operators. Next the H_{xx,yy} term may be written

$$\text{?}$$

$$\text{?} \text{indicates text missing or illegible when filed}$$

where in the last step, a basis rotation 908 on qubit pairs {p,q} is executed that diagonalizes those terms to Zₚ-Z_{q} 910. In effect, this allows to measure the XX and YY terms simultaneously for each pair, and to rotate to a diagonal basis where the particle number should have been maintained. If this is done for N/2 distinct pairs of the total N modes, effectively all modes in the particle-basis are measured and can therefore filter out some of the noisy bitstring measurements.

In FIG. 9C, post-selection error mitigation results are shown for a particular example of LiH molecule in STO-3G basis set at equilibrium bond distance. With increasing noise but no error mitigation (graph 914), very poor performance is found even with low 10⁻³ readout error rates. At a readout error of 10⁻², doing post-selection only on the originally-diagonal terms yields a large increase in accuracy down to almost 1 kcal/mol (graph 916). Next, all XX+YY terms are rotated to the particle basis and perform post-selection on the results, further improvement is shown (graph 918). For the same accuracy of 1 kcal/mol, the additional error mitigation allows for a 45% higher readout error rate.

1. A method for simulating a quantum chemistry system using a data processing system comprising a classical computer connected to a quantum computer, the method comprising:
receiving or determining, by the classical computer, information on a Hamiltonian describing the quantum chemistry system, the Hamiltonian being a hard core bosonic Hamiltonian, which restricted to electron singlet state configurations;
receiving or determining, by the classical computer, information on a paired-electron unitary coupled cluster with double excitations (pUCCD) ansatz, the ansatz being restricted to electron singlet state configurations;
transforming, by the classical computer, the pUCCD ansatz into a quantum circuit, the quantum circuit representing a sequence of qubit operations;
executing, by the quantum computer, the quantum circuit, the execution including applying the sequence of gate operations to qubits of the quantum computer:
receiving, by the classical computer, a trial state, the trial state including measured expectation values of the Hamiltonian; and,
determining an energy of the quantum chemistry system based on the trial state.

2. The method according to claim 1, wherein the electron singlet state configurations only include molecular orbitals that are either occupied or not occupied by electron pairs, preferably a qubit includes a first qubit state |1 [symbol] representing a molecular orbital that is occupied with an electron pair and a second qubit state |0 [symbol] representing a molecular orbital that is not occupied with an electron pair.

3. The method according to claim 1 wherein the Hamiltonian describing the quantum chemistry system is defined in terms of hard-core bosonic annihilation operators, preferably according to the following equation:
$$\text{?} = C + {\sum }_{p , q} {h}_{p , q}^{( r 1 )} \text{?} + {\sum }_{p \neq q} {h}_{p , q}^{( r 2 )} \text{?}$$
$$\text{?} \text{indicates text missing or illegible when filed}$$
or, wherein the Hamiltonian describing the quantum chemistry system is defined in terms of in terms of Pauli spin operators, preferably according to the following equation:
$$\text{?}$$
$$\text{?} \text{indicates text missing or illegible when filed}$$

4. The method according to claim 1 wherein a paired-electron unitary coupled cluster with double excitations (pUCCD) ansatz is defined in terms of annihilation operators, preferably according to the following equation:
$$\hat{U} = exp ( {T}_{s} ) \\ {\hat{T}}_{s} = {\sum }_{i ∈ v , j ∈ o} {t}_{i , j}^{( s )} ( {\hat{b}}_{i}^{†} {\hat{b}}_{j} - {\hat{b}}_{j}^{†} {\hat{b}}_{i} )$$
or, wherein a paired-electron unitary coupled cluster with double excitations (pUCCD) ansatz is defined in terms of Pauli spin operators, preferably according to the following equation:
$${\hat{T}}_{qb} = {\sum }_{i ∈ v , j ∈ o} {t}_{i , j}^{( s )} \text{?} / 2 ( {\hat{σ}}_{p}^{X} {\hat{σ}}_{p}^{Y} - {\hat{σ}}_{p}^{Y} {\hat{σ}}_{p}^{X} ) \text{?}$$
$$\text{?} \text{indicates text missing or illegible when filed}$$

5. The method according to claim 1 wherein, the trial state and the energy are determined based on a variational scheme, preferably a variational quantum eigensolver (VQE) scheme.

6. The method according to claim 5, wherein determining a trial state includes:
initializing the qubits of the quantum computer based on parameters, preferably coupled cluster amplitudes, which are computed on the basis of information of the quantum chemistry system.

7. The method according to claim 5, wherein determining a trial state includes:
sequentially applying gate operations of the quantum circuit to pairs of qubits, an application of a gate causing the pair of qubits to interact with each other.

8. The method according to 7, wherein determining a trial state further includes:
applying a basis rotation to each of the qubits; and, performing one or more qubit readout, a qubit readout representing one of the measured expectation values of the Hamiltonian.

9. The method according to claim 5 wherein, the trial state is error-mitigated using a diagonalize-and-post-selection procedure, filtering out non-physical measurement results (errors).

10. The method according to claim 5 wherein the gate operation is a singlet-state simulation (SSS) gate operation, including a partial-swap gate operation, partial-swap gate performs an entangling operation which simulates partially distributing an electron pair among two molecular orbitals.

11. The method according to claim 5 wherein the gate operation is a singlet-state simulation (SSS) gate operation, including a full-swap gate, which swaps qubit labels in order to bring every qubit which was occupied next to every other qubit which was not occupied.

12. The method according to claim 1, wherein the trial state and the energy are determined based on a quantum phase estimation (QPE) scheme.

13. A system for simulating a quantum chemistry system using a data processing system comprising a classical computer connected to a quantum computer, the system comprising:
a computer readable storage medium having computer readable program code embodied therewith, and a processor, preferably a microprocessor, coupled to the computer readable storage medium, wherein responsive to executing the computer readable program code, the processor is configured to perform executable operations comprising:
configuring a data processing system comprising a classical computer connected to a quantum computer;
receiving or determining, by the classical computer, information on a Hamiltonian describing the quantum chemistry system, the Hamiltonian being a hard core bosonic Hamiltonian, which restricted to electron singlet state configurations;
receiving or determining, by the classical computer, information on a paired-electron unitary coupled cluster with double excitations (pUCCD) ansatz, the ansatz being restricted to electron singlet state configurations;
transforming, by the classical computer, the pUCCD ansatz into a quantum circuit, the quantum circuit representing a sequence of qubit operations;
executing, by the quantum computer, the quantum circuit, the execution including applying the sequence of gate operations to qubits of the quantum computer;
receiving, by the classical computer, a trial state, the trial state including measured expectation values of the Hamiltonian; and,
determining an energy of the quantum chemistry system based on the trial state.

14. The system according to claim 13 wherein the electron singlet state configurations only include molecular orbitals that are occupied or not occupied by electron pairs, preferably a qubit includes a first qubit state |1 [symbol] representing a molecular orbital that is occupied with an electron pair and a second qubit state |0 [symbol] representing a molecular orbital that is not occupied with an electron pair.

15. A computer program product comprising one or more computer-readable storage devices, and program instructions stored on at least one of the one or more storage devices, the stored program instructions comprising instructions for:
configuring a data processing system comprising a classical computer connected to a quantum computer;
receiving or determining, by the classical computer, information on a Hamiltonian describing the quantum chemistry system, the Hamiltonian being a hard core bosonic Hamiltonian, which restricted to electron singlet state configurations;
receiving or determining, by the classical computer, information on a paired-electron unitary coupled cluster with double excitations (pUCCD) ansatz, the ansatz being restricted to electron singlet state configurations;
transforming, by the classical computer, the pUCCD ansatz into a quantum circuit, the quantum circuit representing a sequence of qubit operations;
executing, by the quantum computer, the quantum circuit, the execution including applying the sequence of gate operations to qubits of the quantum computer;
receiving, by the classical computer, a trial state, the trial state including measured expectation values of the Hamiltonian; and,
determining an energy of the quantum chemistry system based on the trial state.
